(19) United States
(12) Patent Application Publication
(10) Pub. No.: US 2024/0412866 A1
(43) Pub. Date: Dec. 12, 2024

(54) OMNI-CHANNEL ARTIFICIAL INTELLIGENCE (AI) CHATBOT FOR MEDICAL DIAGNOSIS AND MEDICAL TREATMENT

(71) Applicant: INTERNATIONAL BUSINESS MACHINES CORPORATION, Armonk, NY (US)

(72) Inventors: Bo Wen, New York, NY (US); Vince Siu, Thornhill (CA); Chen Wang, Chappaqua, NY (US); Hongfei Tian, North Haledon, NJ (US)

(21) Appl. No.: 18/333,404

(22) Filed: Jun. 12, 2023

(51) Int. Cl. G16H 50/20; H04L 51/02

(52) U.S. Cl. CPC G16H 50/20; H04L 51/02

(57) ABSTRACT

Provided are techniques for an omni-channel Artificial Intelligence (AI) chatbot module collecting data and performing analysis. Survey questions for a participant are received. A first channel of a plurality of channels of communication and a period of time for contact are identified. A conversation is initiated by attempting to contact the participant using the first channel and during the period of time. in response to the participant accepting the contact, each of the survey questions are converted to natural language. There is interaction with the participant using the first channel to receive survey answers to the survey questions in the natural language. The survey answers are analyzed, and an analysis result is output.

### BACKGROUND

Embodiments of the invention relate to an omni-channel Artificial Intelligence (AI) chatbot collecting data and performing analysis for medical diagnosis and medical treatment of a medical condition.

A cohort may be described as a group of people with one or more shared characteristics. For example, one cohort may be patients who have limited access to computers or prefer verbal communication to surveys or questionnaires. Past clinical trial analysis showed low compliance for this cohort to finish printed or computer displayed surveys or questionnaires. However, it is important to collect information from this cohort to provide them with better care.

Some clinical studies have clinic staff make phone calls to these patients to collect information, but it is a costly way of collecting that information. Recently, there has been a shortage of clinic staff, and many clinical studies are facing problems as there are not enough clinic staff to make the phone calls.

### SUMMARY

In accordance with certain embodiments, a computer-implemented method comprising operations is provided for an omni-channel AI chatbot module. In such embodiments, survey questions for a participant are received. A first channel of a plurality of channels of communication and a period of time for contact are identified. A conversation is initiated by attempting to contact the participant using the first channel and during the period of time. in response to the participant accepting the contact, each of the survey questions are converted to natural language. There is interaction with the participant using the first channel to receive survey answers to the survey questions in the natural language. The survey answers are analyzed, and an analysis result is output.

In accordance with other embodiments, a computer program product comprising a computer readable storage medium having program code embodied therewith is provided, where the program code is executable by at least one processor to perform operations for an omni-channel AI chatbot module. In such embodiments, survey questions for a participant are received. A first channel of a plurality of channels of communication and a period of time for contact are identified. A conversation is initiated by attempting to contact the participant using the first channel and during the period of time. in response to the participant accepting the contact, each of the survey questions are converted to natural language. There is interaction with the participant using the first channel to receive survey answers to the survey questions in the natural language. The survey answers are analyzed, and an analysis result is output.

In accordance with yet other embodiments, a computer system comprises one or more processors, one or more computer-readable memories and one or more computer-readable, tangible storage devices; and program instructions, stored on at least one of the one or more computer-readable, tangible storage devices for execution by at least one of the one or more processors via at least one of the one or more memories, to perform operations for an omni-channel AI chatbot module. In such embodiments, survey questions for a participant are received. A first channel of a plurality of channels of communication and a period of time for contact are identified. A conversation is initiated by attempting to contact the participant using the first channel and during the period of time. in response to the participant accepting the contact, each of the survey questions are converted to natural language. There is interaction with the participant using the first channel to receive survey answers to the survey questions in the natural language. The survey answers are analyzed, and an analysis result is output.

### BRIEF DESCRIPTION OF THE SEVERAL VIEWS OF THE DRAWINGS

Referring now to the drawings in which like reference numbers represent corresponding parts throughout:

FIG. 1 illustrates a computing environment in accordance with certain embodiments.

FIG. 2 illustrates, in a block diagram, a computing environment with an omni-channel AI chatbot in accordance with certain embodiments.

FIG. 3 illustrates a contextual diagram of an omni-channel AI chatbot in accordance with certain embodiments.

FIG. 4 illustrates, in a flowchart, operations for participant enrollment into a clinical study in accordance with certain embodiments.

FIG. 5 illustrates, in a flowchart, operations for generating survey questions in accordance with certain embodiments.

FIGS. 6A and 6B illustrate, in a flowchart, operations for data collection and analysis in accordance with certain embodiments.

FIG. 7 illustrates an architecture for data collection and analysis in accordance with certain embodiments.

FIG. 8 illustrates, in a flowchart, operations for an omni-channel AI chatbot collecting data and performing analysis in accordance with certain embodiments.

FIG. 9 illustrates, in a block diagram, details of a machine learning model in accordance with certain embodiments.

### DETAILED DESCRIPTION

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

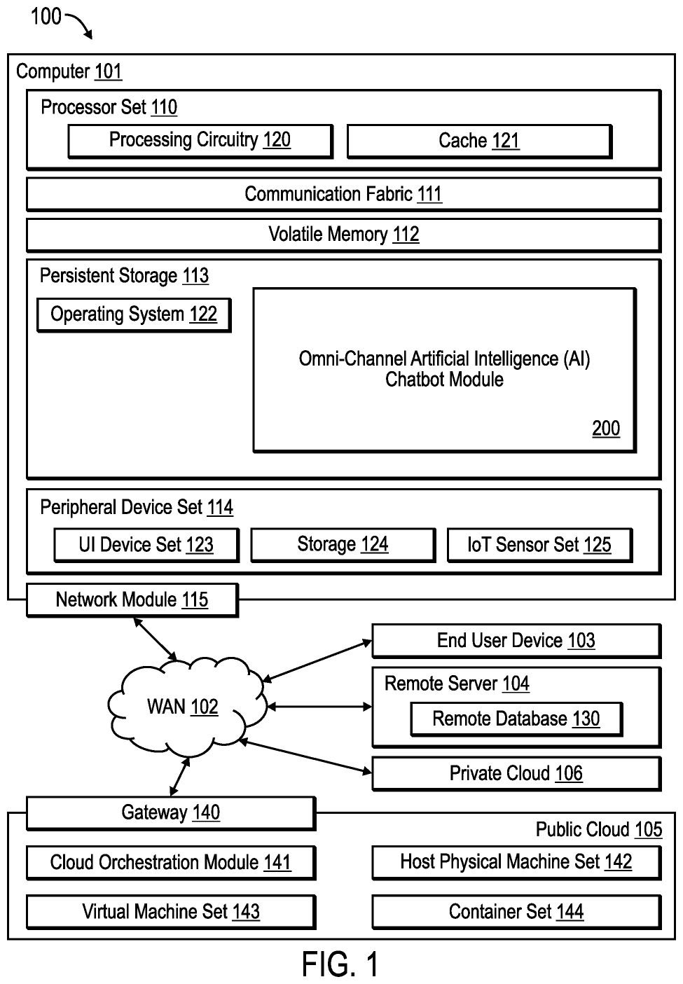

FIG. 1 illustrates a computing environment 100 in accordance with certain embodiments. Computing environment 100 contains an example of an environment for the execution of at least some of the computer code involved in performing the inventive methods, such as an omni-channel AI chatbot module 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115.

Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods.

In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

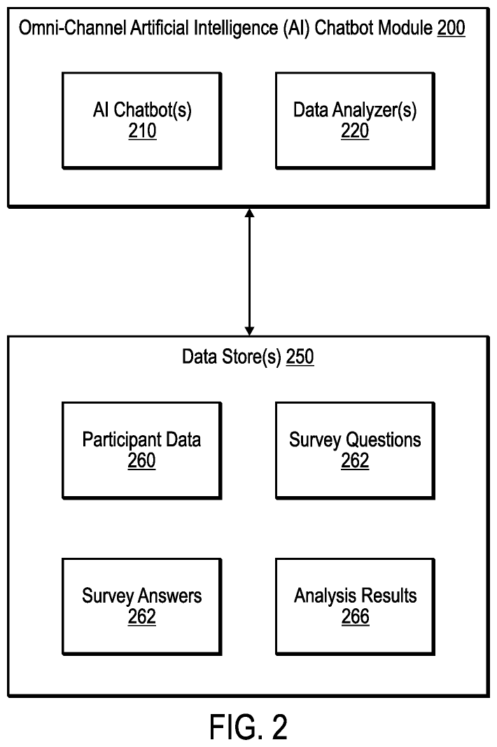

Embodiments provide an omni-channel AI chatbot (i.e., a computer assisted technologies) to reduce the burden on clinic staff and improve operational efficiency. FIG. 2 illustrates, in a block diagram, a computing environment with an omni-channel AI chatbot module 200 in accordance with certain embodiments. The omni-channel AI chatbot module 200 collects and analyzes data. The omni-channel AI chatbot module 200 includes one or more AI chatbot 210s and one or more data analyzers 220. The omni-channel AI chatbot module 200 is connected to one or more data stores 250. The one or more data stores 250 store participant data 260, survey questions 262, survey answers 264, and analysis results 266.

In certain embodiments, the AI chatbot 210 performs the operations of the data analyzer 220. In other embodiments, the AI chatbot 210 sends a request for analysis to the data analyzer 220 and receives analysis results 266. The omni-channel AI chatbot module 200 may use any AI chatbot 210 and data analyzer 220, which allows for customizing the omni-channel AI chatbot module 200. In certain embodiments, the AI chatbot 210 is a machine learning model that is trained on medical knowledge and clinical staff interactions with patients. In certain embodiments, a pair of an AI chatbot 210 and a data analyzer 220 are associated with a particular disease and trained for that disease, such that different pairs of the AI chatbot 210 and the data analyzer 220 may be selected and initiated for different clinical studies. In certain embodiments, the data analysis 220 performs one or more downstream analytic tasks.

In certain embodiments, based on a Large Language Model (LLM) or a foundational model, the AI chatbot 210 may be trained as a generic language generator, and the specific medical knowledge may be supplied to the AI chatbot 210 during runtime through prompts (i.e., through prompt engineering) (e.g., based on the patient/disease type, medical knowledge is retrieved from a database and supplied to the AI chatbot 210.

The omni-channels may be described as multiple channels of communication (i.e., a plurality of channels of communication), such as by phone calls (“voice”), by text messages (“text”), by video calls, via social media, web application, etc. The participant may be a patient participating in the clinical study to obtain, for example, a medical diagnosis and a medical treatment (e.g., an indication of which medicines to take) of a medical condition.

In certain embodiments, the participant data 260 may be a participant profile for a participant and may store a phone number, preference data (e.g., preferences for which omni-channels to use for contact and a time associated with each of the omni-channels), participant name, which of one or more cohorts the participant belongs to, etc. For example, the preference data may indicate an order of omni-channels to try and associated acceptable times to call (e.g., text messages between 5 pm and 8 pm, phone calls between 7 pm and 9 pm, etc.). The preference data may also be referred to as participant preference data or participant preference settings.

In certain embodiments, the analysis results 266 may be one or more of: a medical diagnosis and a medical treatment for a participant, a recommendation of other data to collect from the participant, transcription by Automatic Speech Recognition ASR, clinical information extraction, Natural Language Processing (NLP), acoustic analysis for cognitive decline and neurodegenerative disease assessments, etc.

In certain embodiments, the omni-channel AI chatbot module 200 uses one or more AI chatbots 210 to make phone/video calls to participants or converse with them via text messages to collect data (e.g., survey answers to survey questions and other healthcare-related information). The omni-channel AI chatbot module 200 reduces the clinic staff's burden and streamlines storage and management of the collected data for downstream analytic tasks. In certain embodiments, each AI chatbot 210 is capable of mimicking an actual human's natural language skill, and, if the AI chatbot 210 is able to mimic a human's natural language skill, this improves participant compliance and engagement with the clinical study. Natural language may be described as a language that has evolved naturally as a means of communication among people (as contrasted with an artificial language or computer code). In certain embodiments, the omni-channel AI chatbot module 200 outperforms passive data collection techniques (e.g., an online survey form).

In certain embodiments, the omni-channel AI chatbot module 200 provides an AI chatbot 210 and a data analyzer 220 for clinical data management. The AI chatbot 210 is able to make phone/video calls or engage in a text message-based chat with a clinical study participant based on a preferred schedule, hold a conversation with the participant, obtain information and survey answers to survey questions determined by the clinical study and approved by an internal review board. The participants' responses are recorded and saved in a secure data vault. The audio/video recording or text conversations may be used for various downstream analyses.

The omni-channel AI chatbot module 200 uses the AI chatbot 210 with omni-channels (voice, video, text message, video calls, social media, web application, etc.) as a frontend to interact with participants for clinical study data collection. The AI chatbot 210 access a body of medical knowledge and mimics clinical staff to interact with participants.

Many medical questionnaires have survey questions with multiple choices that use medical terms and jargon. The AI chatbot 210 asks the survey questions in natural language and, optionally, in the participant's preferred language (e.g., a native spoken language) and collects the survey answers in the natural language (which may be the same or different from the natural language of the survey questions). The AI chatbot 210 is able to convert between languages (e.g., between English, Spanish, Mandarin, etc.). In certain embodiments, the AI chatbot 210 has access to a mapping from and medical terms and jargon to natural language terms in one or more languages. In other embodiments, the AI chatbot 210 is trained with this conversion knowledge. Then, the AI chatbot 210 converts each survey answer (i.e., speech content from the participant) into an embedding using NLP models, retrieves the embedding of each multiple choice answer, calculates the cosine similarity of the survey answer embedding with the multiple choice answer embedding, and selects the closest multiple choice answer based on the closeness of the survey answer embedding and the multiple choice answer embedding. In this manner, the omni-channel AI chatbot module 200 lowers the communication barrier for the participants to provide responses to medical survey questions, which also encourages participation and improves compliance.

In natural language processing, an embedding may be described as a representation of a word, a sentence or a paragraph. In certain embodiments, the embedding may be a vector (matrix) representation that is easier for neural networks to process. In certain embodiments, an embedding is a string of numbers that serves as a unique identifier, and that string of numbers is a real-valued vector that encodes the meaning of the word in such a way that words that are closer in the vector space are expected to be similar in meaning.

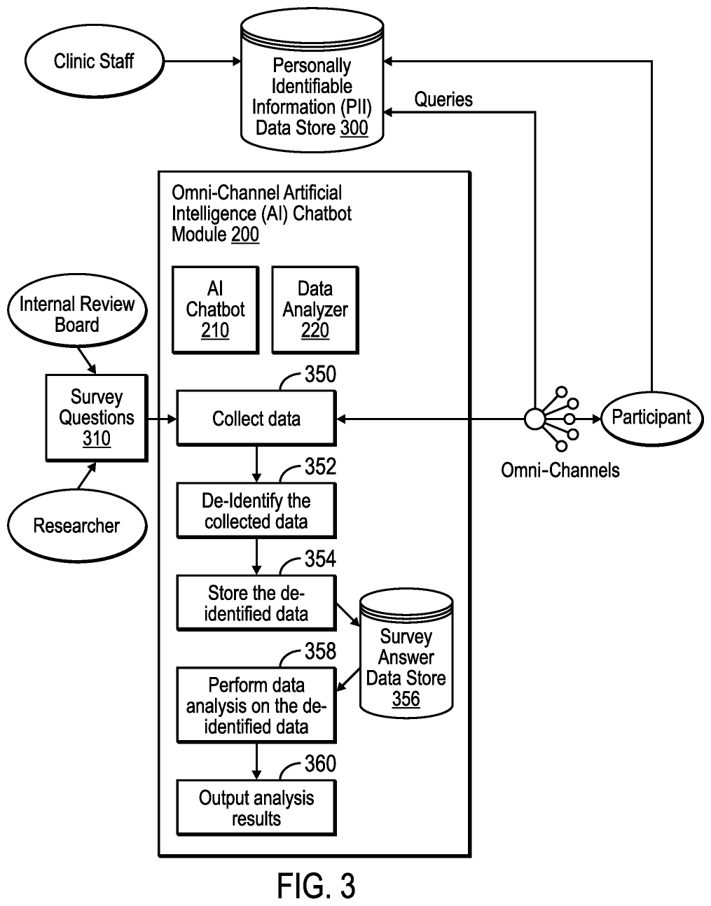

FIG. 3 illustrates a contextual diagram of an omni-channel AI chatbot module 200

in accordance with certain embodiments. In FIG. 3, clinic staff and/or participants in a clinical study store participant data in a Personally Identifiable Information (PII) data store 300 (e.g., a database). The PII data that is stored in the PII data store 300 is sensitive information, such as patient names, phone numbers, addresses, etc. The PII data is guarded in an environment compliant to regulations to protect such data. Thus, in certain embodiments, the AI chatbot 210 does not store the PII data (for security of the PII). Instead, the AI chatbot 210 queries the PII data store 300 to get the PII data.

In addition researcher may create survey questions 310 (i.e., a question set) for the clinical study, and a review board determines whether to approve the survey questions. The approved survey questions are stored for the AI chatbot 210 to retrieve. The AI chatbot 210 retrieves (or receives) the survey questions 310 and communicates with the participant using one or more of the omni-channels to obtain survey answers to the survey questions 310. In this manner, the AI chatbot 210 collects data (block 350). The AI chatbot 210 de-identifies the collected data (block 352) to remove any data that identifies the participant to maintain the participant's privacy. The AI chatbot 210 stores the de-identified data (block 354) in a survey answer data store 356 (e.g., a database).

In certain embodiments, the AI chatbot 210 queries (using one of the omni-channels) the PII data store 300 for a phone number or other contact information to contact the participant. The AI chatbot 210 makes a connection (using one of the omni-channels) with the participate (e.g., with a phone call or a text message). The AI chatbot 210 sends (using one of the omni-channels) collected survey answers to the backend system.

Then, the data analyzer 220 retrieves the de-identified data from the survey answer data store 356 and performs data analysis (block 358). The data analyzer 220 outputs analysis results 266.

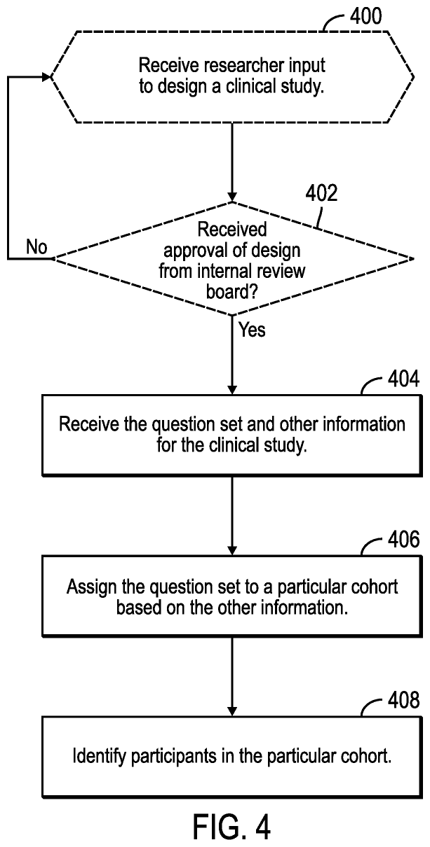

FIG. 4 illustrates, in a flowchart, operations for generating survey questions in accordance with certain embodiments. Control begins at block 400 with receipt of researcher input to design a clinical study. The researcher input may include survey questions and other information (e.g., defining one or more cohorts) for the clinical study. In block 402, it is determined whether the internal review board approves the design for the clinical study. If so, processing continues to block 404, otherwise, processing loops back to block 400 so that the survey questions may be modified.

In block 404, the omni-channel AI chatbot module 200 receives the question set for the clinical study. In block 406, the omni-channel AI chatbot module 200 assigns the question set to a particular cohort (i.e., group of participants that share one or more characteristics) based on the other information. In block 408, the omni-channel AI identifies participants in the particular cohort. This may be done by accessing a data store that provides, for each participant, one or more cohorts that the participant belongs to.

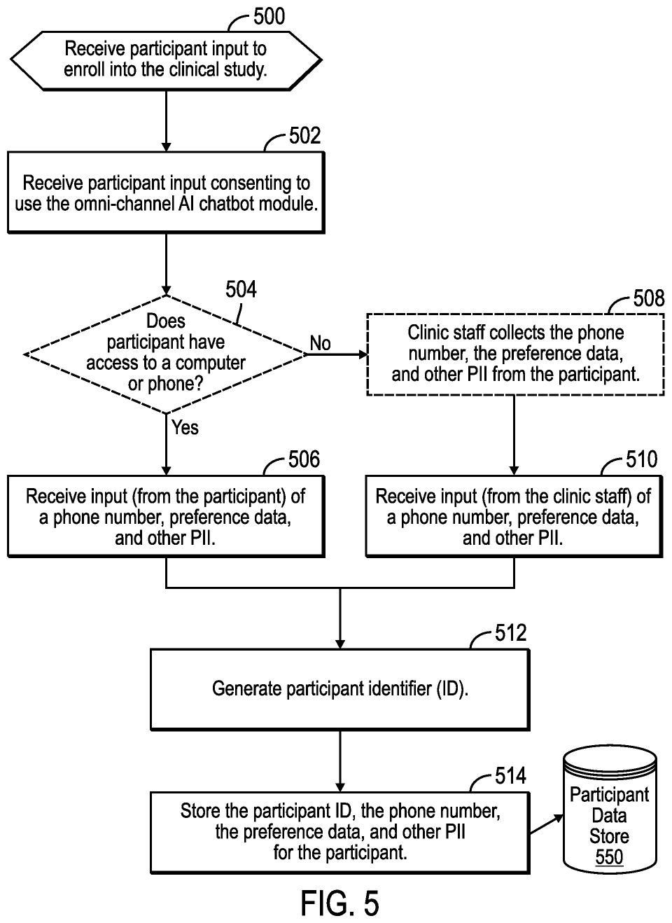

FIG. 5 illustrates, in a flowchart, operations for participant enrollment into a clinical study in accordance with certain embodiments. Control begins at block 500 with the omni-channel AI chatbot module 200 receiving participant input to enroll into the clinical study. For example, the participant is one of the participants identified in block 408 for a particular cohort for a clinical study. In block 502, the omni-channel AI chatbot module 200 receives participant input consenting to use the omni-channel AI chatbot. The participant input of blocks 500 and 502 may be via a user interface of the omni-channel AI chatbot module 200, may be provided to the omni-channel AI chatbot module 200 from a clinic system that collected the participant input via signature on a document or via a web application.

In block 504, the omni-channel AI chatbot module 200 determines whether the participant has access to a computer or phone (e.g., based on participant input). If so, processing continues to block 506, otherwise, processing continues to block 508.

In block 506, the omni-channel AI chatbot module 200 receives input (from the participant) of a phone number (e.g., of a landline telephone or of the smart phone), preference data (e.g., preferences for which omni-channels to use for contact and a time associated with each of the omni-channels), and other information (e.g., PII, such as a name (e.g., so that the chatbot may address the participant by name in communications)). The AI chatbot module 200 may receive the participant input via a smart phone (e.g., fora phone call, a text message or a video call, a landline phone (e.g., for a phone call) or a computer (e.g., for social media or web applications).

In certain embodiments, the participant may be at a clinic or lab and may want to provide input to the clinic staff. Therefore, in block 508, the clinic staff collects the phone number, the preference data, and other PII from the participant (e.g., via a phone call to a land line)

In block 510, the omni-channel AI chatbot module 200 receives input (from the clinic staff) of a phone number, preference data (e.g., preferences for which omni-channels to use for contact and a time associated with each of the omni-channels), and other information (e.g., PII, such as a name (e.g., so that the chatbot may address the participant by name in communications)).

From blocks 506 and 510, processing continues to block 512. In block 512, the omni-channel AI chatbot module 200 generates participant identifier (ID). In block 514, the omni-channel AI chatbot module 200 stores the participant ID, the phone number, preference data, and the other PII for the participant in a participant data store 550 (e.g., an example of participant data 260, which may be a database). In certain embodiments, the participant ID, the phone number, preference data, and the other PII may be stored in the PII data store 300 instead of or in addition to the participant data store 550.

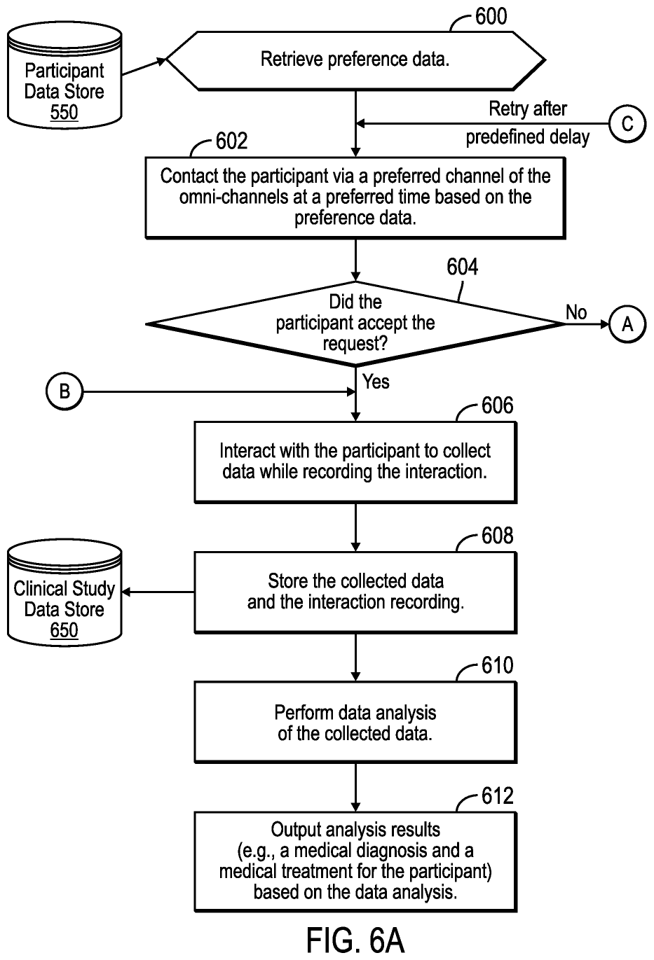

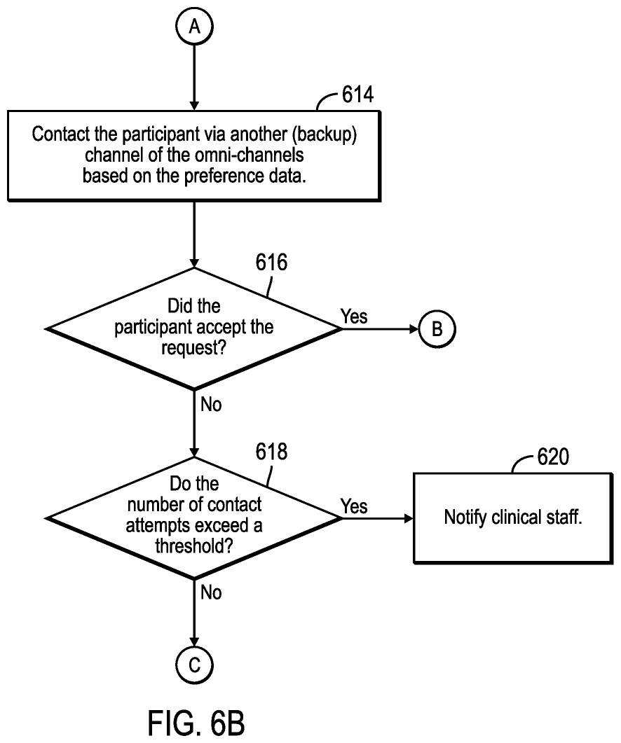

FIGS. 6A and 6B illustrate, in a flowchart, operations for data collection and analysis in accordance with certain embodiments. Control begins at block 600 with the AI chatbot 210 of the omni-channel AI chatbot module 200 retrieving preference data from a participant data store 550. In certain embodiments, the preference data may also be stored in the PII data store 300. In block 602, the AI chatbot 210 contacts the participant via a preferred channel of the omni-channels at a preferred time based on the preference data. In block 604, the AI chatbot 210 determines whether the participant accepted the request (e.g., answered a phone call, responded to a text, etc.). If so, processing continues to block 606, otherwise, processing continues to block 614 (FIG. 6B).

In block 606, the AI chatbot 210 interacts with the participant to collect data while recording the interaction. This may include retrieving survey questions for the clinical study, converting the survey questions into a preferred language of the participant and/or into a more understandable format (e.g., by adding definitions of medical terms used in the survey questions). This may also include, for each survey question that has multiple choice answers, receiving a survey answer 264 in natural language form, matching the survey answer 264 to the closest multiple choice answer, and selecting that multiple choice answer.

In block 608, the AI chatbot 210 stores the collected data and the interaction recording. In block 610, the data analyzer 220 of the omni-channel AI chatbot module 200 performs data analysis of the collected data. In block 612, the data analyzer 220 outputs analysis results 266 based on the data analysis.

In block 614 (FIG. 6B), the AI chatbot 210 contacts the participant via another (e.g., a backup) channel of the omni-channels based on the preference data. In block 616, the AI chatbot 210 determines whether the participant accepted the request. If so, processing continues to block 606 (FIG. 6A), otherwise, processing continues to block 618. In block 618, the AI chatbot 210 determines whether the number of contact attempts exceed a threshold. If so, processing continues to block 620, otherwise, processing continues to block 602 to attempt contact again after a pre-defined delay. That is, the AI chatbot 210 attempts to contact the participant a predetermined number of attempts. In certain embodiments, the participant data 260 also stores the predefined dela.

In block 620, the AI chatbot 210 notifies the clinic staff that the participant could not be contacted.

Merely to enhance understanding of embodiments, examples are provided herein.

In a first example, for many chronic conditions, a patient's symptoms vary from day to day, and it is useful for doctors to have a way to monitor the patients' symptoms. Unlike wearable and Internet of Things (IoT) solutions that may not be affordable, the omni-channel AI chatbot module 200 works with patients who have access to a telephone, which allows the omni-channel AI chatbot module 200 to be permissive. Doctors and clinical staff may assign appropriate follow-up inquiries to the omni-channel AI chatbot module 200. For example, the omni-channel AI chatbot module 200 may be given survey questions specific for Crohn's disease, survey questions for diagnosing and treating depression (e.g., by providing a prescription for medication or sending the prescription to a pharmacy to be filled), and/or survey questions to monitor patient-reported health status for physical, mental, and social well-being. The omni-channel AI chatbot module 200 then contacts the patient (who has provided consent and agreement to participate), asks the survey questions, and records the survey answers according to a periodic schedule that is appropriate for the situation.

In certain embodiments, the omni-channel AI chatbot module 200, in real time, matches the survey answers to a medical diagnosis and a medical treatment (e.g., in a database or using rules). Thus, the omni-channel AI chatbot module 200 efficiently provides a new medical treatment (e.g., a new prescription medication) or updates a current medical treatment (e.g., changing doses of an existing medication taken by the participant). In certain embodiments, the omni-channel AI chatbot module 200 orders the new or updated prescription to be mailed to the participant.

As a second example, for pharmaceutical clinical studies, the omni-channel AI chatbot module 200 is able to check on new medicine's treatment effectiveness. In particular, the omni-channel AI chatbot module 200 contacts patients at a specific time of a day to ask whether they have taken the new medicine on time and how they feel. With a natural language capability, the AI chatbot 210 may improve patient's engagement and compliance.

In certain embodiments, the omni-channel frontend allows the omni-channel AI chatbot module 200 to interact with patients not only via phone calls (voice), but also text messages, video calls, social media or web applications.

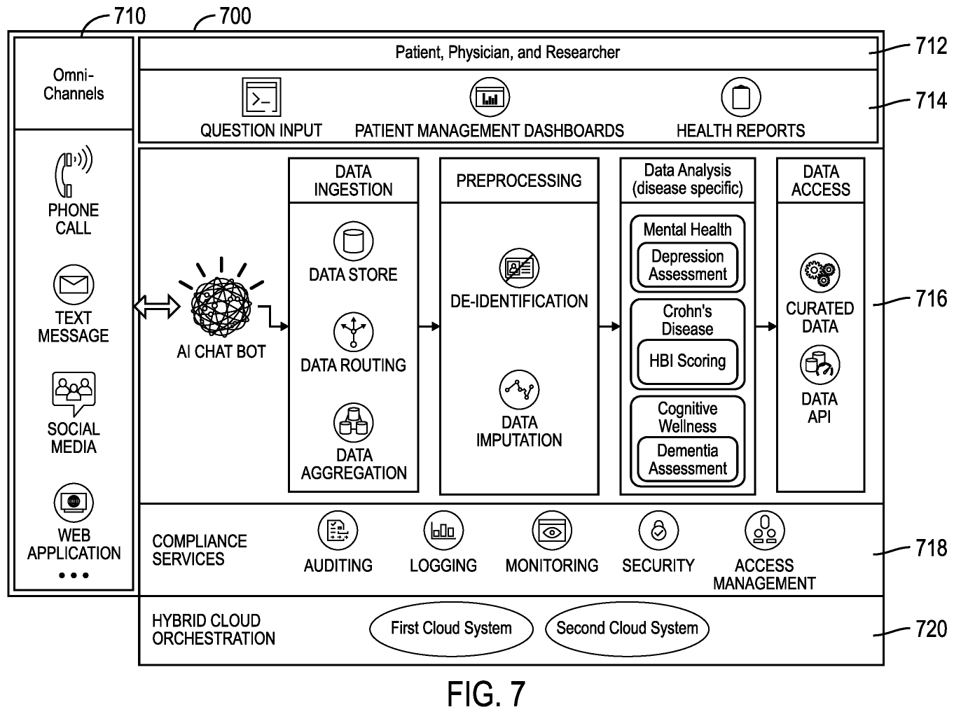

FIG. 7 illustrates an architecture 700 for data collection and analysis in accordance with certain embodiments. Within the architecture 700, there are omni-channel data sources 710 (e.g., phone call, text message, video call, social media, web application, etc.). There are also patient, physician, and researcher interfaces 712. The architecture 700 allows for question input, patient management dashboards, and health reports 714. The data collection and analysis components 716 are an example of the omni-channel AI chatbot module 200 and include the AI chatbot contacting a patient through one or more of the omni-channels to ingest the data, pre-process the data, perform data analysis, and allow data access. The data ingestion allows storing data in a data store, routing data, and aggregating data. The preprocessing allows for de-identification and data imputation (which substitutes missing values with valid values for processing the data). The data analysis is disease specific for this example, and includes but is not limited to checks for mental health (with a depressing assessment), Crohn's disease (with Harvey-Bradshaw Index (HBI) scoring), and cognitive wellness (with dementia assessment). The data access provides curated data and proves a data Application Programming Interface (API). The architecture 700 includes compliance services of auditing, logging, monitoring, security, and access management. The architecture 700 supports hybrid cloud orchestration, and the omni-channel AI chatbot module 200 may run on one or multiple cloud systems.

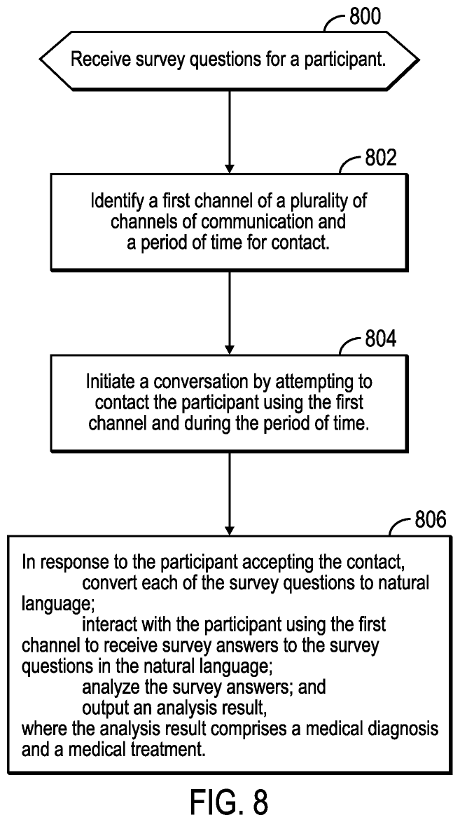

FIG. 8 illustrates, in a flowchart, operations for an omni-channel AI chatbot module 200 collecting data and performing analysis in accordance with certain embodiments. The operations of FIG. 8 are performed under control of the omni-channel AI chatbot module 200. Control begins at block 800 with the omni-channel AI chatbot module 200 receiving survey questions for a participant. In block 802, the omni-channel AI chatbot module 200 identifies a first channel of a plurality of channels of communication and a period of time for contact. In block 804, the omni-channel AI chatbot module 200 initiates a conversation by attempting to contact the participant using the first channel and during the period of time. In block 806, in response to the participant accepting the contact, the omni-channel AI chatbot module 200 converts each of the survey questions to natural language, interacts with the participant using the first channel to receive survey answers to the survey questions in the natural language, analyzes the survey answers, and outputs an analysis result.

Thus, the omni-channel AI chatbot module 200 reduces the clinic staff burden and streamlines the data storage and analysis for research and clinical operations. The omni-channel AI chatbot module 200 improves patient compliance and engagement. In addition, the omni-channel AI chatbot module 200 avoids providing medical suggestions, which avoids the risk of providing inaccurate medical information.

The omni-channel AI chatbot module 200 allows the data controller (e.g., the clinical study group) to be a separate entity and provides data privacy (e.g., de-identification) and compliance management components.

In certain embodiments, the omni-channel AI chatbot module 200 collects patient information by providing an AI chatbot 210 trained on medical knowledge and clinical staff interactions with patients to provide survey questions in natural language and receive survey answers in natural language from patients. In addition, the omni-channel AI chatbot module 200 enables a conversation between the AI chatbot 210 and a patient to obtain information and survey answers to survey questions determined by a clinical study. The AI chatbot 210 initiates the conversation using a channel of omni-channels and a preferred time and date provided by the patient. The omni-channel AI chatbot module 200 records patient survey answers to the AI chatbot and analyzes the survey answers using a data analyzer 220.

In certain embodiments, the AI chatbot 210 converts a clinical study question into natural language and converts the survey answer received from the patient into survey answer embeddings using NLP models. Then, the AI chatbot 210 compares the cosine similarity of the survey answer embeddings with multiple choice answer embeddings associated with the survey question, and the AI chatbot 210 selects the multiple choice answer that has embedding closest to the survey answer embeddings. The AI chatbot 210 records the selected multiple choice answer as the survey answer for the survey question.

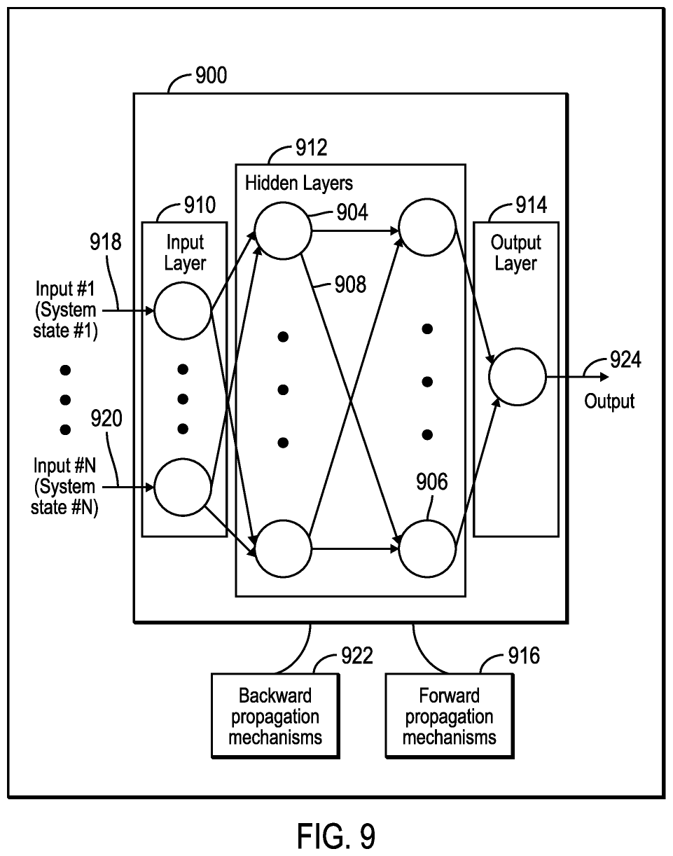

FIG. 9 illustrates, in a block diagram, details of a machine learning model 900 in accordance with certain embodiments. In certain embodiments, one or more AI chatbots 210 are implemented using the components of the machine learning model 900.

The machine learning model 900 may comprise a neural network with a collection of nodes with links connecting them, where the links are referred to as connections. For example, FIG. 9 shows a node 904 connected by a connection 908 to the node 906. The collection of nodes may be organized into three main parts: an input layer 910, one or more hidden layers 912, and an output layer 914.

The connection between one node and another is represented by a number called a weight, where the weight may be either positive (if one node excites another) or negative (if one node suppresses or inhibits another). Training the machine learning model 900 entails calibrating the weights in the machine learning model 900 via mechanisms referred to as forward propagation 916 and backward propagation 922. Bias nodes that are not connected to any previous layer may also be maintained in the machine learning model 900. A bias may be described as an extra input of 1 with a weight attached to it for a node.

In forward propagation 916, a set of weights are applied to the input data 918 . . . 920 to calculate the output 924. For the first forward propagation, the set of weights may be selected randomly or set by, for example, a system administrator. That is, in the forward propagation 916, embodiments apply a set of weights to the input data 918 . . . 920 and calculate an output 924.

In backward propagation 922 a measurement is made for a margin of error of the

output 924, and the weights are adjusted to decrease the error. Backward propagation 922 compares the output that the machine learning model 900 produces with the output that the machine learning model 900 was meant to produce, and uses the difference between them to modify the weights of the connections between the nodes of the machine learning model 900, starting from the output layer 914 through the hidden layers 912 to the input layer 910, i.e., going backward in the machine learning model 900. In time, backward propagation 922 causes the machine learning model 900 to learn, reducing the difference between actual and intended output to the point where the two come very close or coincide.

The machine learning model 900 may be trained using backward propagation to adjust weights at nodes in a hidden layer to produce adjusted output values based on the provided inputs 918 . . . 920. A margin of error may be determined with respect to the actual output 924 from the machine learning model 900 and an expected output to train the machine learning model 900 to produce the desired output value based on a calculated expected output. In backward propagation, the margin of error of the output may be measured and the weights at nodes in the hidden layers 912 may be adjusted accordingly to decrease the error.

Backward propagation may comprise a technique for supervised learning of artificial neural networks using gradient descent. Given an artificial neural network and an error function, the technique may calculate the gradient of the error function with respect to the artificial neural network's weights.

Thus, the machine learning model 900 is configured to repeat both forward and backward propagation until the weights of the machine learning model 900 are calibrated to accurately predict an output.

The machine learning model 900 implements a machine learning technique such as decision tree learning, association rule learning, artificial neural network, inductive programming logic, support vector machines, Bayesian models, etc., to determine the output value 924.

In certain machine learning model 900 implementations, weights in a hidden layer of nodes may be assigned to these inputs to indicate their predictive quality in relation to other of the inputs based on training to reach the output value 924.

With embodiments, the machine learning model 900 is a neural network, which may be described as a collection of “neurons” with “synapses” connecting them.

With embodiments, there may be multiple hidden layers 912, with the term “deep” learning implying multiple hidden layers. Hidden layers 912 may be useful when the neural network has to make sense of something complicated, contextual, or non-obvious, such as image recognition. The term “deep” learning comes from having many hidden layers. These layers are known as “hidden”, since they are not visible as a network output.

In certain embodiments, training a neural network may be described as calibrating all of the “weights” by repeating the forward propagation 916 and the backward propagation 922.

In backward propagation 922, embodiments measure the margin of error of the output and adjust the weights accordingly to decrease the error.

Neural networks repeat both forward and backward propagation until the weights are calibrated to accurately predict the output 924.

In certain embodiments, the inputs to the machine learning model 900 are survey questions 262 and survey answers 264, and the outputs of the machine learning model 900 are analysis results 266.

The terms “an embodiment”, “embodiment”, “embodiments”, “the embodiment”, “the embodiments”, “one or more embodiments”, “some embodiments”, and “one embodiment” mean “one or more (but not all) embodiments of the present invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

In the described embodiment, variables a, b, c, i, n, m, p, r, etc., when used with different elements may denote a same or different instance of that element.

Devices that are in communication with each other need not be in continuous communication with each other, unless expressly specified otherwise. In addition, devices that are in communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the present invention need not include the device itself.

The foregoing description of various embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, embodiments of the invention reside in the claims herein after appended. The foregoing description provides examples of embodiments of the invention, and variations and substitutions may be made in other embodiments.

What is claimed is:

1. A computer-implemented method, comprising operations for:
under control of an omni-channel Artificial Intelligence (AI) chatbot module,
receiving survey questions for a participant;
identifying a first channel of a plurality of channels of communication and a period of time for contact;
initiating a conversation by attempting to contact the participant using the first channel and during the period of time; and
in response to the participant accepting the contact,
converting each of the survey questions to natural language;
interacting with the participant using the first channel to receive survey answers to the survey questions in the natural language;
analyzing the survey answers; and
outputting an analysis result, wherein the analysis result comprises a medical diagnosis and a medical treatment.

2. The computer-implemented method of claim 1, wherein the omni-channel AI chatbot comprises an AI chatbot that is trained on medical knowledge and clinical staff interactions with other participants.

3. The computer-implemented method of claim 1, wherein the plurality of channels of communication comprise phone calls, text messages, video calls, social media, and web applications.

4. The computer-implemented method of claim 1, further comprising operations for:
in response to the participant rejecting the contact, attempting to contact the participant using one or more other channels of the plurality of channels of communication for a predetermined number of attempts.

5. The computer-implemented method of claim 1, wherein an order of channels of the plurality of channels of communication is stored for the participant with a corresponding period of time for contact for each of the channels.

6. The computer-implemented method of claim 1, further comprising operations for:
converting a survey answer of the survey answers received from the participant into a survey answer embedding using a Natural Language Processing (NLP) model;
comparing the survey answer embedding with embeddings of a plurality of multiple choice answers associated with the survey question; and
selecting a multiple choice answer from the plurality of multiple choice answers having an embedding that is closest to the survey answer embedding.

7. The computer-implemented method of claim 1, wherein the analysis result is selected from a group comprising: the medical diagnosis and the medical treatment for the participant, an effectiveness of the medical treatment for the participant, a recommendation of other data to collect from the participant, transcription by Automatic Speech Recognition (ASR), clinical information extraction, Natural Language Processing (NLP), and acoustic analysis for cognitive decline and neurodegenerative disease assessments.

8. A computer program product, the computer program product comprising a computer readable storage medium having program instructions embodied therewith, the program instructions executable by a processor to cause the processor to perform operations for:
under control of an omni-channel Artificial Intelligence (AI) chatbot module,
receiving survey questions for a participant;
identifying a first channel of a plurality of channels of communication and a period of time for contact;
initiating a conversation by attempting to contact the participant using the first channel and during the period of time; and
in response to the participant accepting the contact,
converting each of the survey questions to natural language;
interacting with the participant using the first channel to receive survey answers to the survey questions in the natural language;
analyzing the survey answers; and
outputting an analysis result, wherein the analysis result comprises a medical diagnosis and a medical treatment.

9. The computer program product of claim 8, wherein the omni-channel AI chatbot comprises an AI chatbot that is trained on medical knowledge and clinical staff interactions with other participants.

10. The computer program product of claim 8, wherein the plurality of channels of communication comprise phone calls, text messages, video calls, social media, and web applications.

11. The computer program product of claim 8, wherein the program instructions are executable by the processor to cause the processor to perform operations for:
in response to the participant rejecting the contact, attempting to contact the participant using one or more other channels of the plurality of channels of communication for a predetermined number of attempts.

12. The computer program product of claim 8, wherein an order of channels of the plurality of channels of communication is stored for the participant with a corresponding period of time for contact for each of the channels.

13. The computer program product of claim 8, wherein the program instructions are executable by the processor to cause the processor to perform operations for:
converting a survey answer of the survey answers received from the participant into a survey answer embedding using a Natural Language Processing (NLP) model;
comparing the survey answer embedding with embeddings of a plurality of multiple choice answers associated with the survey question; and
selecting a multiple choice answer from the plurality of multiple choice answers having an embedding that is closest to the survey answer embedding.

14. The computer program product of claim 8, wherein the analysis result is selected from a group comprising: the medical diagnosis and the medical treatment for the participant, an effectiveness of the medical treatment for the participant, a recommendation of other data to collect from the participant, transcription by Automatic Speech Recognition (ASR), clinical information extraction, Natural Language Processing (NLP), and acoustic analysis for cognitive decline and neurodegenerative disease assessments.

15. A computer system, comprising:
one or more processors, one or more computer-readable memories and one or more computer-readable, tangible storage devices; and
program instructions, stored on at least one of the one or more computer-readable, tangible storage devices for execution by at least one of the one or more processors via at least one of the one or more computer-readable memories, to perform operations comprising:
under control of an omni-channel Artificial Intelligence (AI) chatbot module,
receiving survey questions for a participant;
identifying a first channel of a plurality of channels of communication and a period of time for contact;
initiating a conversation by attempting to contact the participant using the first channel and during the period of time; and
in response to the participant accepting the contact,
converting each of the survey questions to natural language;
interacting with the participant using the first channel to receive survey answers to the survey questions in the natural language;
analyzing the survey answers; and
outputting an analysis result, wherein the analysis result comprises a medical diagnosis and a medical treatment.

16. The computer system of claim 15, wherein the omni-channel AI chatbot comprises an AI chatbot that is trained on medical knowledge and clinical staff interactions with other participants.

17. The computer system of claim 15, wherein the plurality of channels of communication comprise phone calls, text messages, video calls, social media, and web applications.

18. The computer system of claim 15, wherein the program instructions further perform operations comprising:
in response to the participant rejecting the contact, attempting to contact the participant using one or more other channels of the plurality of channels of communication for a predetermined number of attempts.

19. The computer system of claim 15, wherein an order of channels of the plurality of channels of communication is stored for the participant with a corresponding period of time for contact for each of the channels.

20. The computer system of claim 15, wherein the program instructions further perform operations comprising:
converting a survey answer of the survey answers received from the participant into a survey answer embedding using a Natural Language Processing (NLP) model;
comparing the survey answer embedding with embeddings of a plurality of multiple choice answers associated with the survey question; and
selecting a multiple choice answer from the plurality of multiple choice answers having an embedding that is closest to the survey answer embedding.
